(19) United States
(12) Patent Application Publication
(10) Pub. No.: US 2026/0355307 A1
(43) Pub. Date: Nov. 12, 2026

(54) PLASMA MEMBRANE-PERMEABILIZED INACTIVATED ORAL VACCINE

(71) Applicant: NINGBO MINGYI BIOTECHNOLOGY CO., LTD., Ningbo, Zhejiang (CN)

(72) Inventor: Yiwei JIANG, Ningbo, Zhejiang (CN)

(21) Appl. No.: 19/123,724

(22) PCT Filed: Oct. 18, 2023
(86) PCT No.: PCT/CN2023/125248

§ 371 Date: Apr. 23, 2025

(30) Foreign Application Priority Data
Oct. 25, 2022 (CN) 202211310154.1

(51) Int. Cl. A61K 39/39; A61K 39/00; A61K 39/02; A61K 39/17; A61P 37/04

(52) U.S. Cl. CPC A61K 39/39; A61K 39/107; A61K 39/17; A61P 37/04; A61K 2039/542; A61K 2039/552; A61K 2039/55594

(57) ABSTRACT

The present invention provides a plasma membrane-permeabilized inactivated oral vaccine and a preparation method therefor. The inactivated oral vaccine includes a pathogen inactivated by permeabilization by a permeabilizer, a plasma membrane, and an integral membrane protein thereof, wherein the pathogen is selected from the group consisting of an enveloped virus, a bacterium, a fungus, an endoparasite, and a cancer cell, and the permeabilizer includes a lipopeptide as a surfactant.

### CROSS REFERENCE TO RELATED APPLICATIONS

This application is a U.S. National Phase Application of International Application No. PCT/CN2023/125248, filed Oct. 18, 2023, which claims priority to Chinese Patent Application No. 202211310154.1 filed Oct. 25, 2022. The entire contents of all of the above-mentioned applications are incorporated herein by reference into this disclosure.

### SEQUENCE LISTING

The instant application contains a Sequence Listing which has been submitted electronically in XML file format and is hereby incorporated by reference in its entirety. Said XML copy, created on Apr. 16, 2025, is named P25H11405A-US01-seq and is 25,844 bytes in size.

### TECHNICAL FIELD

The present disclosure relates generally to biotechnology and, more particularly, to inactivated oral vaccines prepared by permeabilization of plasma membranes, and the use thereof.

### BACKGROUND OF THE DISCLOSURE

Vaccines are extremely important to the health of humans and non-human animals, and there is a continuous need for new or better vaccines. However, the development of vaccines is challenging and needs to meet the often opposing requirements of both safety and effectiveness. The advantage of attenuated live vaccines is that they can stimulate both humoral immunity and cellular immunity through an almost natural infection process. However, live attenuated vaccines may cause diseases in individuals in immunosuppressed states.

In contrast, conventional inactivated vaccines avoid the risk of infection by pathogens and associated diseases, but they suffer from other drawbacks. First of all, even with adjuvants, conventional inactivated vaccines can only stimulate humoral immunity, but basically fail to stimulate cellular immunity. Therefore, the protection is weaker and shorter-lived, necessitating higher doses and more frequent administration. Secondly, the commonly used injection delivery methods for inactivated vaccines may bring new problems in safety and effectiveness. Since inactivated vaccines cannot break through the biological barrier of the host via natural infection and elicit an immune response, most inactivated vaccines can only be delivered by injection, but the pain and panic caused by the injection can lead to a stress response, which can induce immunosuppression and then weaken the immune response, ultimately leading to a suboptimal vaccination outcome. In addition, the injection of veterinary inactivated vaccines usually requires capturing and restraining the animals. These operations will further intensify the stress response and affect the vaccination outcome.

Oral administration is the safest and most convenient manner for vaccine delivery. More importantly, oral vaccines can stimulate cellular immunity and humoral immunity at both the mucosal level and body level, thus constructing multiple lines of defense against infection, and intercepting microbial pathogens outside the body by effective mucosal immunity. However, inactivated oral vaccines face the challenge of complete loss of antigens due to degradation by digestive enzymes in the gastrointestinal tract. Therefore, the form or dosage form of inactivated oral vaccine antigens must possess the ability to resist digestion and be efficiently absorbed. How to protect antigens and make them cross the gastrointestinal tract barrier efficiently and be transported to immune cells is the key to oral inactivated vaccines.

Natural absorption of antigens in the gut provides a technical inspiration for oral vaccine antigen delivery. Microfold cells (or M cells) are highly differentiated cells in the epithelium covering organized lymphoid follicles of the small intestine and large intestine. M cells sample diverse molecules, recognize proteins, and transport antigens by delivering bacteria, viruses, parasites, and non-infectious particles to the underlying lymphoid tissue, the Peyer's patches. Based on their central role in initiating mucosal immune responses, M cells have become the delivery target of artificial vaccine carriers that allow antigens to cross the cellular barrier of the gastrointestinal tract. Taking advantage of the property that M cells prefer to take up particles containing a structure of lipid bilayer membrane, artificial encapsulation technology using lipid materials has been developed to protect and deliver oral vaccine antigens. However, the preparation of artificial carrier vaccines with M cells as direct delivery targets, such as liposome and bilosome vaccines, involves complex processes and high costs, limiting their application to “high-end vaccines.”

In order to avoid or reduce the waste of immune resources caused by the competition of ineffective and low-efficiency antigens, it is necessary to separate and purify high-efficient antigens during conventional inactivated vaccine preparation. For example, the antigen preparation procedure of an inactivated influenza vaccine involves (i) inoculating a selected strain into embryonated chicken eggs for amplification, (ii) purifying the virus, (iii) dual inactivation with formaldehyde and β-propiolactone, (iv) removing inactivating agents, and (v) isolating the highly effective antigens with a detergent. However, conventional antigen purification is incompatible with the technical requirements of inactivated oral vaccines. After purification, the “naked” purified antigens are easily degraded and rendered inactive by digestive enzymes in the gastrointestinal tract, while encapsulation protection would involve complex processes and high costs.

Therefore, there is a need in the art for oral vaccines with simple manufacturing processes and low costs.

### SUMMARY OF THE INVENTION

To address the deficiencies of the prior art, the present disclosure provides an oral vaccine with simple manufacturing processes and low costs, as well as preparation method and use of the vaccine. In the oral vaccines of the present disclosure, pathogens or attenuated vaccines thereof are inactivated by permeabilizing the plasma membrane with a permeabilizer, and can be prepared without conventional steps such as antigen isolation (e.g., virus) and/or artificial antigen encapsulation.

The present disclosure is based on an unexpected discovery made by the present inventors. Contrary to the teaching of the prior art (see Chinese Patent Application No. CN201810354453.2), which states that BT lipopeptides and vaccines should be delivered separately, the inventor found that BT lipopeptides do not cause the failure of immunization by live attenuated vaccines when they are orally administered together with the live attenuated vaccines. Furthermore, the present inventors have unexpectedly found that when using a permeabilizer with the BT lipopeptide as the only surfactant ingredient to inactivate a pathogen, oral intake such BT lipopeptide-inactivated vaccine provides much higher protection than oral vaccination with a high-temperature inactivated vaccine.

In one aspect, the present disclosure provides an inactivated oral vaccine comprising a pathogen or a live vaccine thereof inactivated by permeabilization of the plasma membrane with a permeabilizer, wherein the inactivated pathogen or live vaccine thereof comprises a permeabilized plasma membrane and its integral membrane protein, wherein the pathogen is a pathogen comprising plasma membrane, and the permeabilizer comprises a lipopeptide as a surfactant.

In one embodiment, the pathogen is selected from the group consisting of an enveloped virus, a bacterium, a fungus, an endoparasite, and a cancer cell.

In one embodiment, the plasma membrane and its integral membrane protein are the native plasma membrane and its integral membrane protein of the pathogen or live vaccine thereof. In one embodiment, the permeabilizer permeabilizes only the bilayer membrane of phospholipid molecules. In one embodiment, the permeabilizer only permeabilizes the bilayer membrane of phospholipid without altering the bilayer membrane structure of the phospholipid molecules. In one embodiment, the permeabilizer does not detach the integral membrane protein from the phospholipid, maintaining the natural conformation of the integral membrane protein.

In one embodiment, the vaccine further comprises the exuded contents of the pathogen or live vaccine thereof after the permeabilization.

In one embodiment, the vaccine is inactivated by a step comprising plasma membrane-permeabilization of the pathogen or the live vaccine thereof.

In one embodiment, the step of permeabilization of the plasma membrane results in the number of live pathogens less than 10¹/ml and/or the number of dead pathogens greater than 10⁹/ml.

In one embodiment, the inactivated oral vaccine is an oral solution, a powder, a tablet, a capsule, a drench solution, or an oral bolus solution.

In one embodiment, the inactivated oral vaccine is a vaccine administered by direct oral administration, water-based delivery, feed-based delivery, gavage, or oral bolus injection.

In one embodiment, the permeabilizer contains no surfactants except the lipopeptide.

In one embodiment, the lipopeptide is the sole surfactant.

In one embodiment, the lipopeptide comprises a natural lipopeptide and/or an artificially synthesized lipopeptide.

In one embodiment, the natural lipopeptide is selected from Surfactins, Fengycins, Iturins, Daptomycin, Polymyxins, Viscosin, and a lipopeptide produced from Brevibacillus texasporus.

In one embodiment, the lipopeptide produced from B. texasporus comprises lipopeptides having the sequence set forth in any one of SEQ ID NOs: 1-9 or any combination thereof.

In one embodiment, the pathogen is selected from the group consisting of: Coronaviridae, Influenza viruses, Paramyxoviridae, Herpesviridae, Poxviridae, Hepadnaviridae, Baculoviruidae, Retroviridae, African swine fever virus, Acinetobacter, Bacilli, Borrelia, Campylobacter, Clostridia, Vibrio, E. coli, Enterococcus, Neisseria, Haemophilus, Mannheimia, Mycobacteria, Mycoplasma, Pasteurellaceae, Pseudomonas, Salmonella, Staphylococcus, Streptococcus, Coccidia, Nematoda, Cestoda, and cancer cells.

In one embodiment, the pathogen is selected from the group consisting of Newcastle disease virus and Vibrio parahaemolyticus.

In one embodiment, the inactivated oral vaccine comprises a pharmaceutically acceptable carrier, or comprises an ingredient that modulates immunity or promotes health or growth, such as an adjuvant, or a lipopeptide produced from B. texasporus. In one embodiment, the lipopeptide produced from B. texasporus comprises a lipopeptide(s) having the sequence set forth in any one of SEQ ID NOs: 1-9 or any combination thereof.

In another aspect, provided is a method for preparing the inactivated oral vaccine described herein, comprising:
permeabilizing the plasma membrane of the pathogen or live vaccine thereof, wherein the permeabilizer comprises a lipopeptide as a surfactant.

In one embodiment, the method does not comprise a step of isolating the antigen and/or a step of artificially encapsulating the antigen. For example, the method does not include the step of packaging or encapsulating the antigen in a liposome or bilosome.

In one embodiment, when the pathogen is inactivated, the method does not comprise inactivation means other than permeabilization, such as high temperature inactivation.

In one embodiment, the permeabilizer contains no surfactants except the lipopeptide.

In one embodiment, the plasma membrane of the pathogen or live vaccine thereof is permeabilized, such that the number of live pathogens is less than 10¹/ml and/or the number of dead pathogens is greater than 10⁹/ml.

In one embodiment, the lipopeptide comprises a natural lipopeptide and/or an artificially synthesized lipopeptide.

In one embodiment, the natural lipopeptide is selected from the group consisting of Surfactins, Fengycins, Iturins, Daptomycin, Polymyxins, Viscosin, and the lipopeptide produced from Brevibacillus texasporus.

In one embodiment, the lipopeptide produced from B. texasporus comprises a lipopeptide(s) having the sequence set forth in any one of SEQ ID NOs: 1-9 or any combination thereof.

In yet another aspect, the present disclosure provides an oral composition comprising the inactivated oral vaccine described herein.

In one embodiment, the oral composition comprises food, drinking water, or beverage.

In one embodiment, the food is selected from the group consisting of a milk substitute, growth feed, finisher feed, pre-starter feed, and starter feed.

In one embodiment, the composition comprises one or more of cereal, soybean flour, isolated soybean protein, soybean oil, soybean fat, skim milk, fish meal, meat meal, bone meal, blood meal, plasma protein, whey, rice bran, wheat bran, sweeteners, minerals, vitamins, and salts.

In one embodiment, the cereal is selected from the group consisting of barley, soybean, wheat, triticale, rye, corn, and rice.

In one embodiment, the oral composition comprises an ingredient that modulates immunity or promotes health or growth, preferably a BT lipopeptide which is mixed with the inactivated oral vaccine or is isolated. For example, the BT lipopeptide includes a BT lipopeptide(s) produced from B. texasporus, preferably a BT lipopeptide(s) having the sequence set forth in any one of SEQ ID NOs: 1-9 or any combination thereof. The oral composition may comprise a BT lipopeptide, which is mixed with or separated from the inactivated oral vaccine. The concentration of the BT lipopeptide may be 100 ppm or more, such as 150 ppm, 200 ppm, 250 ppm, 300 ppm, 330 ppm, 400 ppm, 450 ppm, 500 ppm, 550 ppm, 600 ppm, 650 ppm, 700 ppm, 750 ppm, 800 ppm, 850 ppm, 900 ppm, or 950 ppm.

In yet another aspect, the present disclosure provides the use of a permeabilizer in the manufacture of the inactivated oral vaccine. This use is accomplished by perforating the plasma membrane of the pathogen or the live vaccine thereof to cause exudation of the contents inside the plasma membrane, thus inactivating the pathogen or live vaccine thereof; wherein the permeabilizer comprises a lipopeptide as a surfactant.

In one embodiment, the inactivated oral vaccine is an inactivated oral vaccine described herein.

In one embodiment, the lipopeptide comprises a natural lipopeptide and/or an artificially synthesized lipopeptide.

In one embodiment, the natural lipopeptide is selected from the group consisting of Surfactins, Fengycins, Iturins, Daptomycin, Polymyxins, Viscosin, and lipopeptides produced from Brevibacillus texasporus.

In one embodiment, the lipopeptide produced from B. texasporus comprises lipopeptide having the sequence set forth in any one of SEQ ID NOs: 1-9 or any combination thereof.

In yet another aspect, the present disclosure provides the use of the inactivated oral vaccine described herein in the manufacture of a medicament for treating and/or preventing an infection or disease caused by a pathogen in a subject.

In yet another aspect, the present disclosure provides a method for treating and/or preventing an infection or disease caused by a pathogen in a subject, comprising administration to the subject the inactivated oral vaccine described herein.

In yet another aspect, provided is an inactivated oral vaccine described herein for use in treating and/or preventing an infection or disease caused by a pathogen in a subject.

In one embodiment of the above aspects, the infection or disease is selected from the group consisting of influenza, viral hepatitis, African swine fever, porcine reproductive and respiratory syndrome, colibacillosis, salmonellosis, necrotic enteritis, vibrosis, bovine shipping fever pneumonia, Gonorrhea, urinary tract infection, coccidiosis, nematodiasis, cestodiasis, and cancer.

In yet another aspect, the present disclosure provides use of the inactivated oral vaccine described herein in the manufacture of a feed for improving bodyweight gain and/or food conversion in a subject.

In yet another aspect, the present disclosure provides a method for improving bodyweight gain and/or food conversion in a subject, comprising administration to the subject of the inactivated oral vaccine described herein.

In one embodiment of the above aspects, the subject is a bird, a livestock, an aquatic animal, or a companion animal. In one embodiment of the above aspects, the feed is applied by in ovo feeding, spraying in the form of droplets onto a bird or immersion of an aquatic animal.

The advantageous effects of the present disclosure include: (1) the inactivated oral vaccine described herein is orally delivered to the gastrointestinal tract, where the digestive enzymes naturally degrade the exuded contents to remove potential intramembrane antigen molecules, thereby selectively retaining the perforated plasma membrane and its integral membrane protein as antigen molecules, which are ultimately absorbed to induce mucosal and systemic immune responses, completely eliminating the antigen purification steps and their costs during the manufacture of the inactivated vaccine; (2) The inactivated oral vaccines described herein do not require additional protective treatments for the antigen, such as treatments that protect the antigen from degradation in the gastrointestinal tract (e.g., liposome-encapsulation, etc.).

### DETAILED DESCRIPTION OF THE DISCLOSURE

### DEFINITION

In order to facilitate the understanding of the present disclosure, a number of terms are defined below. The terms defined herein have the meanings commonly understood by one of ordinary skill in the art to which the present disclosure relates.

As used herein, “an animal” means any vertebrate or invertebrate, including, but not limited to, a human, a horse, a goat, a sheep, a cow, a pig, a chicken, a turkey, a hen, a goose, a duck, a dog, a cat, a parrot, a fish, a crab, a shrimp (e.g. Penaeus vannamei), freshwater turtles and other animals.

As used herein, “attenuated” refers to reducing the ability of a virus, a bacterium, a fungus or a parasite to cause a disease in an animal.

As used herein, a “live vaccine” refers to a live microbial preparation made by artificially attenuating a pathogen or by screening avirulent or minimally virulent strains of a pathogen from nature, sometimes referred to as a live attenuated vaccine.

As used herein, “inactivated” refers to a virus, a bacterium, a fungus, a parasite, or a cancer cell irreversibly loses the ability to replicate or proliferate. An “inactivator” refers to a substance or energy that irreversibly deprives a virus, a bacterium, a fungus, a parasite, or a cancer cell of the ability to replicate or proliferate.

As used herein, “plasma membrane permeabilization” and “plasma membrane perforation” are synonymous, both referring to the impairment of plasma membrane integrity leading to content exudation, without altering the overall bilayer structure of membrane phospholipid molecules, nor changing the conformation of integral membrane proteins or their binding to the phospholipid bilayer.

As used herein, “plasma membrane dissolution” refers to the conversion of a phospholipid molecule-bilayer into a phospholipid miscelle, while the integral membrane proteins are detached from the phospholipid and denatured.

As used herein, a “surfactant” refers to an amphipathic substance that reduces the surface tension of interfaces such as those between liquids. Different types of surfactants have different types of destructive effects on the plasma membrane structure. Detergent surfactants dissolve the plasma membrane, while lipopeptide surfactants permeabilize but do not dissolve the plasma membrane.

The lipopeptide surfactant may include natural and an artificially synthesized lipopeptide surfactants. Natural lipopeptides are secondary metabolites produced by microorganisms. They generally have one long lipid chain and 7 to 25 amino acid residues. Natural lipopeptides have been shown to have anti-bacterial, anti-viral, anti-fungal, anti-tumor, and immunomodulatory functions. Natural lipopeptide surfactants include, but are not limited to, Surfactins, Fengycins, Iturins, Daptomycin, Polymyxins, Viscosin, and BT lipopeptides.

As used herein, “BT lipopeptide” may refer to a single lipopeptide or a mixture of multiple lipopeptides produced from Brevibacillus laterosporus texasporus, in particular, a single lipopeptide or a mixture of multiple lipopeptides comprising the sequences of SEQ ID NOs: 1-9 as disclosed in CN201380056514.2 and CN201810354453.2. Brevibacillus texasporus (e.g., ATCC PTA-5854) is a previously identified soil bacterium that expresses a non-ribosomal peptide synthetase (NRPS, encoded by the operon under GenBank accession number AY953371) to produce a family/mixture of related cationic NRP variants of 13 amino acid residues in length (“BT peptides” or “BT lipopeptides” in light of their newly resolved N-terminal structure, and the two terms are interchangeable in the present disclosure), among which BT1583 is the most abundant variant (WO/2005/074626). The cationic peptides (as a mixture or an individual peptide isolated from B. texasporus) display a broad-spectrum antibacterial activity in vitro (BT Function #1). Based on the high degree of 16S rDNA sequence identity (98.5%) between PTA-5854 and Brevibacillus laterosporus type strain, Brevibacillus texasporus is classified as a subspecies of Brevibacillus laterosporus, with Brevibacillus laterosporus subsp. texasporus being defined as Brevibacillus laterosporus strains that produce the nonribosomal peptides from the BT NRPS (or BT peptides). Genomic sequencing of at least two B. laterosporus strains (LMG 15441 and GI-9) has validated this taxonomy. Both genomes (published respectively under GenBank accession number AFR V00000000 and EMBL accession numbers CAGD01000001 to CAGD01000061) contain an intact BT NRPS operon with 99% DNA sequence identity to AY953371, even though these B. laterosporus strains are not known to be producers of BT peptides. “Brevibacillus texasporus”, “Brevibacillus laterosporus subsp. texasporus” and “B. texasporus” are thus synonymous.

The BT peptide or BT lipopeptide may be a mixture of natural BT NRP variants (including SEQ ID NOs: 1-9) isolated from B. texasporus using the separation and detection methods disclosed in WO/2005/074626 and Wu, X., Ballard, J. and Jiang, Y. W. (2005). Structure and biosynthesis of the BT peptide antibiotic from Brevibacillus texasporus. Appl Environ Microbiol 71, 8519-8530, except that the separation and detection methods do not contain a reverse-phase HPLC step for resolving BT NRP variants. BT peptides or BT lipopeptides can be readily prepared by those skilled in the art using the methods described in these references. BT peptides or BT lipopeptides were characterized, and the amino acid sequences are provided in Table 1 below. Table 1 describes the structures of the nine BT NRP variants and their usual relative abundances in such mixtures.

TABLE 1
Summary of BT NRP variants (Hmp represents 2-hydroxy-3-
methylpentanoic acid, Dhb represents 2, 3-dehydro-2-aminobutyric acid, which is
synonymous with dehydrothreonine (Dht), Vol represents valinol, subscripts D and E
represent D-type chirality and (E)-configuration in amino acids, respectively, and
% indicates the variant relative abundance among BT NRP variants as a mixture
isolated from B. texasporus cells, when determined by mass spectrometry).

| | | | | |
|---|---|---|---|---|
| BT | | | SEQ ID | |
| variant | Peptide sequence | % | NO. | Bogoro1 |

Dominant variant

| | | | | |
|---|---|---|---|---|
| BT1583 | Hmp-EDhb-Leu-_{D}Orn-Ile-Val-Val-_{D}Lys-Val-_{D}Leu- | 31 | 1 | A |
| | Lys-_{D}Tyr-Leu-Vol | | | |

Minority variants

| | | | | |
|---|---|---|---|---|
| BT1601-M2 | Hmp-_{E}Dhb-Met-_{D}Orn-Ile-Val-Val-_{D}Lys-Val-_{D}Leu- | 26 | 2 | D |
| | Lys-_{D}Tyr-Leu-Vol | | | |
| BT1569-V2 | Hmp-EDhb-Val-_{D}Orn-Ile-Val-Val-_{D}Lys-Val-_{D}Leu- | 20 | 3 | B |
| | Lys-_{D}Tyr-Leu-Vol | | | |

Detectable variants

| | | | | |
|---|---|---|---|---|
| BT1597-15 | Hmp-_{E}Dhb-Leu-_{D}Orn-Ile-Ile-Val-_{D}Lys-Val-_{D}Leu- | 6 | 4 | |
| | Lys-_{D}Tyr-Leu-Vol | | | |
| BT1611- | Hmp-_{E}Dhb-Leu-_{D}Lys-Ile-Ile-Val-_{D}Lys-Val-_{D}Leu- | 6 | 5 | |
| K315 | Lys-_{D}Tyr-Leu-Vol | | | |
| BT1555- | Hmp-_{E}Dhb-Val-_{D}Orn-Val-Val-Val-_{D}Lys-Val- | 5 | 6 | C |
| V2V4 | _{D}Leu-Lys-_{D}Tyr-Leu-Vol | | | |
| BT1615- | Hmp-_{E}Dhb-Met-_{D}Orn-Ile-Ile-Val-_{D}Lys-Val-_{D}Leu- | 4 | 7 | |
| M215 | Lys-_{D}Tyr-Leu-Vol | | | |
| BT1583-12 | Hmp-_{E}Dhb-Ile-_{D}Orn-Ile-Val-Val-_{D}Lys-Val-_{D}Leu- | 1 | 8 | |
| | Lys-_{D}Tyr-Leu-Vol | | | |
| BT1617-F2 | Hmp-_{E}Dhb-Phe-_{D}Orn-Ile-Val-Val-_{D}Lys-Val-_{D}Leu- | 1 | 9 | |
| | Lys-_{D}Tyr-Leu-Vol | | | |

As used herein, “feed” or “food”, which is synonymous to each other, refers to any substance or mixture of substances which contains amino acids, antioxidants, carbohydrates, flavoring agents, enzymes, fats, minerals, non-proteinaceous nitrogen products, proteins, vitamins, and/or binders, and may contain granulating agents, coloring agents, foaming agents, and/or flavoring agents, that are manufactured, sold, or provided to an animal (e.g., a livestock or a domesticated animal) or consumed by a human, in order to provide at least a portion of the nutritional requirement of the animal or human, and/or for the purpose of preventing or treating a nutritional condition in the animal. As a vital nutrient, water is an ingredient of feed or food, and water-containing beverages belong to feed or food.

### The Inactivated Oral Vaccine of the Present Disclosure

The present disclosure includes inactivated oral vaccines. The main component of an inactivated oral vaccine may be a plasma membrane-permeabilized or—perforated enveloped virus, bacteria, fungus, endoparasite, or cancer cell. The main component of an inactivated oral vaccine may be a bilayer membrane of phospholipid molecules or isolated substance containing the bilayer membrane of phospholipid molecules, which is isolated from a plasma membrane-permeabilized or perforated enveloped virus, bacteria, fungus, endoparasite, or cancer cell. The antigens of the inactivated oral vaccines may comprise a perforated bilayer membrane of phospholipid molecules and its integral membrane proteins derived from an enveloped virus, a bacterium, a fungus, an endoparasite, or a cancer cell.

The main component of the inactivated oral vaccine of the present disclosure may be an enveloped virus, bacterium, fungus, or endoparasite, or the live attenuated vaccine thereof in which plasma membrane is permeabilized or perforated. The main component of the inactivated oral vaccine may be a bilayer membrane of phospholipid molecules or an isolated substance containing the bilayer membrane of phospholipid molecules, which is isolated from an enveloped virus, a bacterium, a fungus or an endoparasite (or a live vaccine of an enveloped virus, a bacterium, a fungus or an endoparasite) with the plasma membrane perforated. The antigens of inactivated oral vaccines may comprise a perforated bilayer membrane of phospholipid molecules and its integral membrane proteins, which is derived from an attenuated enveloped virus, bacteria, fungus or endoparasite (or live vaccines of enveloped viruses, bacteria, fungus, or endoparasites).

### Method for Preparing the Inactivated Oral Vaccine of the Present Disclosure

The present disclosure includes a method for preparing an inactivated oral vaccine, comprising mixing a pathogen or live vaccine thereof with a permeabilizer so that the plasma membrane is perforated to result in exudation of the contents, thus inactivating the pathogen or live vaccine thereof. The pathogen may be an enveloped pathogen, including an enveloped virus, a bacterium, a fungus, an endoparasite, or a cancer cell. In an exemplary embodiment, the permeabilizer used for inactivation is a lipopeptide surfactant. The lipopeptide surfactant includes natural and artificially synthesized lipopeptide surfactants. Examples of natural lipopeptide surfactants include: Surfactins, Fengycins, Iturins, Daptomycin, Polymyxins, Viscosin, and BT lipopeptides. In a further exemplary embodiment, the permeabilizer used for inactivation is a BT lipopeptide. The BT lipopeptide may comprise one or more of the following: sequences selected from the group consisting of SEQ ID NOs: 1-9, or isolated BT lipopeptides derived from B. texasporus. A BT lipopeptide is a purified water-soluble BT lipopeptide. The BT lipopeptides may be a mixture isolated from B. texasporus. In the present disclosure, a BT lipopeptide can be used as an inactivator and/or adjuvant. In the inactivated oral vaccines of the present disclosure, the concentration of BT lipopeptide is sufficient to allow the number of live pathogens less than 10¹/ml and/or the number of dead pathogens greater than 10⁹/ml. For example, the final concentration of the BT lipopeptide in the inactivated oral vaccine may be 100 ppm or more, such as 100 ppm, 500 ppm, 1000 ppm, 1500 ppm, 2000 ppm, 2500 ppm, 3000 ppm, 3500 ppm, 4000 ppm, 4500 ppm, 5000 ppm, or 5500 ppm.

The method involved in inactivating an oral vaccine described herein may not comprise a step of isolating the antigen and/or a step of artificially encapsulating the antigen. For example, the method does not include the step of packaging or encapsulating the antigen in a liposome or bilosome. In one embodiment, when the pathogen is inactivated, the method includes no inactivation means except permeabilization inactivation, such as high temperature inactivation. In one embodiment, the permeabilizer contains no surfactants except the lipopeptide. In one embodiment, the plasma membrane of the pathogen or live vaccine thereof is permeabilized, such that the number of live pathogens is less than 10¹/ml and/or the number of dead pathogens is greater than 10⁹/ml. In one embodiment, the lipopeptide comprises a natural lipopeptide and/or an artificially synthesized lipopeptide. In one embodiment, the natural lipopeptide is selected from the group consisting of Surfactins, Fengycins, Iturins, Daptomycin, Polymyxins, Viscosin, and lipopeptides produced from Brevibacillus texasporus. In one embodiment, the lipopeptides produced from B. texasporus comprises lipopeptides having the sequence set forth in any one of SEQ ID NOs: 1-9 or any combination thereof. In one embodiment, the lipopeptide produced from B. texasporus is a mixture of lipopeptides having the sequence set forth in SEQ ID NOs: 1-9.

### Dosage Forms and Administration

The present disclosure includes dosage forms of plasma membrane-permeabilized inactivated oral vaccines, including but not limited to an oral solution, a powder, a tablet, a capsule, a drench solution, and an oral bolus solution.

The present disclosure also includes delivery means of the inactivated oral vaccines. The delivery means of the inactivated oral vaccines include, but are not limited to, autonomous direct oral administration, water-based delivery, feed-based delivery, gavage, and oral bolus injection. The delivery means of the inactivated oral vaccine also include, but are not limited to, the following special means: injection of the inactivated vaccine solution into the amniotic sac of avian embryos (in-ovo feeding), and spraying the inactivated vaccine in the form of coarse droplets onto birds (especially chicks) for pecking consumption, and immersing aquatic animals (especially their juveniles) into the inactivated vaccine liquid.

The present disclosure also includes food/feed or drinking water/beverage to which the oral inactivated vaccine is added. In one exemplary embodiment, the plasma membrane-permeabilized inactivated vaccine is incorporated into drinking water or animal feed, such as a cereal-based feed, for example a food/feed containing at least one cereal selected from the group consisting of barley, soybean, wheat, triticale, rye, corn, rice, and combinations thereof. In fact, the present invention can be added to a great variety of feeds. The inactivated vaccine of the present disclosure may be mixed with drinking water or feed for livestock selected from the group consisting of milk substitute, growth feed, finisher feed, pre-starter feed, starter feed, water, and combinations thereof. Examples of the ingredient of food/feed also include cereal, soybean flour, isolated soybean protein, isolated soybean oil, isolated soybean fat, skim milk, fish meal, meat meal, bone meal, blood meal, plasma protein, whey, rice bran, or bran, and may also include sweeteners, minerals, vitamins, and salts.

### Use of the Inactivated Oral Vaccine of the Present Disclosure

The present disclosure further comprises a post-inoculation purification method for enriching high-efficacy plasma membrane surface antigens, including oral delivery of the inactivated oral vaccine described herein. The inactivated oral vaccine described herein is orally delivered to the digestive tract, where digestive enzymes naturally degrade exuded contents to eliminate potential intramembrane antigen molecules, thereby selectively retaining perforated plasma membranes and their integral membrane proteins as antigen molecules, which are ultimately absorbed to elicit both mucosal and systemic immune responses. This will completely eliminate antigen purification steps and associated costs in the inactivated vaccine production process. In one exemplary embodiment, the inactivated oral vaccine containing primarily plasma membrane-permeabilized or perforated enveloped viruses, bacteria, fungi, endoparasites or cancer cells is orally delivered, with digestive enzymes degrading the exuded contents to remove potential intramembrane antigen molecules, thereby selectively retaining the perforated plasma membranes and their integral membrane proteins as the ultimately delivered antigen molecules.

The present disclosure further comprises a method for improving weight gain and feed conversion in growing livestock or companion animals, which is performed by orally administering a plasma membrane-permeabilized inactivated vaccine in an effective amount sufficient to prevent and/or treat microbial infections in the animals.

The present disclosure further comprises a method for preventing and/or treating microbial infections in animals, which is performed by orally administering a plasma membrane-permeabilized inactivated vaccine in an effective amount sufficient to prevent and/or treat microbial infections in the animals.

The present disclosure also relates to compositions and/or methods for preventing and/or treating diseases in animals, comprising orally administration to the animal a plasma membrane-permeabilized inactivated vaccine. For example, the treatment may involve reducing pathogens in the animal. Exemplary pathogens that can be reduced may include, but are not limited to, Coronaviridae, Influenza viruses, Paramyxoviridae, Herpesviridae, Poxviridae, Hepadnaviridae, Baculoviruidae, Retroviridae, African swine fever virus, Acinetobacter, Bacilli, Borrelia, Campylobacter, Clostridia, Vibrio, E. coli, Enterococcus, Neisseria, Haemophilus, Mannheimia, Mycobacteria, Mycoplasma, Pasteurellaceae, Pseudomonas, Salmonella, Staphylococcus, Streptococcus, Coccidia, Nematoda, Cestoda, and cancer cells.

Exemplary diseases that may be prevented and/or treated by the present disclosure include, but are not limited to, Newcastle Disease, influenza, viral hepatitis, African swine fever, porcine reproductive and respiratory syndrome, colibacillosis, salmonellosis, necrotic enteritis, vibrosis, bovine shipping fever pneumonia, Gonorrhea, urinary tract infection, coccidiosis, nematodiasis, cestodiasis, and cancer.

The methods of the invention may further comprise oral administration of an isolated BT peptide. The BT peptide may be administered on the same day as the inactivated oral vaccine of the present disclosure, and/or one or several days after the administration of the inactivated oral vaccine of the present disclosure. The BT peptide can be administered for several consecutive days, such as 2, 3, 4, 5, 6, 7, 8, 9, 10, 11, 12, 13, 14, 15, 16, 17, 18, 19, 20, 21, 22, 23, 24, 25, 26, 27, 28, 29, or 30 consecutive days. Alternatively, in order to economize the amount of used BT peptide, the BT peptide may be administered at intervals (pulse administration), for example, at intervals of 1, 2, 3, 4, 5, 5, 6, 7, 8, 9, 10, 11, 12, 13, 14, 15, 16, 17, 18, 19, 20, 21, 22, 23, 24, 25, 26, 27, 28, 29, or 30 days. In this case, the BT peptide may be administered for 1 or more times, such as 2, 3, 4, or 5 times.

The methods of the disclosure may further comprise oral administration of the BT peptide after combining it with the inactivated oral vaccine of the present disclosure into a single preparation.

Hereinafter, embodiments of the present application will be described in detail in conjunction with Examples. Those skilled in the art will understand that the following Examples are merely for illustrating the present application, and should not be regarded as limiting the scope of the present application. If the specific conditions are not specified in an Example, the conventional conditions or the conditions recommended by the manufacturer shall be followed. If the manufacturer of the reagents or instruments used is not specified, they are conventional products that are commercially available. Unless otherwise indicated, the amounts listed are based on the total weight and are described in parts by weight. This application should not be construed as being limited to the specific Examples described.

### EXAMPLES

The BT lipopeptides in the examples of the present invention were prepared using the methods disclosed in WO/2005/074626 and the reference (Wu, X., Ballard, J., and Jiang, Y. W. (2005). Structure and biosynthesis of the BT peptide antibiotic from Brevibacillus texasporus. Appl Environ Microbiol 71, 8519-8530), and using Brevibacillus texasporus strain PTA-12309 (deposited at the American Type Culture Collection) published in CN201280020898.8. Specifically, B. texasporus strain PTA-12309 cells were cultured in 1 liter of LB medium at 37 °C with shaking for 3 days. The culture was centrifuged at 3,000 rpm for 15 minutes. The supernatant was collected, and 500 grams of ammonium sulfate was added and dissolved. The sample was centrifuged at 3,000×g for 15 minutes. The precipitate was dissolved in 200 ml of distilled water. The solution was then boiled for 15 minutes and cooled on ice. The sample was filtered through a 0.2 μm filter (Nalgene). The filtrate was mixed with 0.2 liters of chloroform at room temperature using a stir bar for 20 minutes. The mixture was separated into two phases by centrifugation at 3,000 rpm for 15 minutes. The organic phase was collected and dried in a vacuum evaporator. A person skilled in the art can readily prepare BT peptides or BT lipopeptides using the methods described in these references. The BT peptides or BT lipopeptides were characterized, and their amino acid sequences are provided in Table 1.

### Example 1

The first example of the present disclosure originated from an operational “error” in farming production—the accidental success of effective immunization when BT lipopeptides were “incorrectly” mixed with attenuated live enveloped virus vaccines for oral administration. As disclosed in CN201810354453.2, BT lipopeptides serve as separate adjuvants delivered through drinking water to animals that received vaccines via injection or spray, meaning the BT lipopeptide adjuvant is spatiotemporally separated from the vaccine. In this “mistaken” case, the conventional delivery method-post-hatching spray vaccination with Newcastle Disease Live Vaccine (La Sota strain, Xinpike, Harbin Pharmaceutical Group Biological Vaccine Co., Ltd.) at the hatchery—was changed to drinking water delivery at a commercial chicken farm when the chicks were 2 days old. Since the original protocol required 100 ppm BT lipopeptides in drinking water on that day, the attenuated live Newcastle disease vaccine and BT lipopeptides were inadvertently mixed together in the drinking water. Remarkably, this batch of chickens showed normal survival rates, health status and growth performance, with completely normal Newcastle disease antibody titers at 25 days of age.

The “conflict” of mixing BT lipopeptide adjuvant with attenuated live Newcastle disease vaccine was not recognized and understood until retrospective analysis of this production batch. A composition of “BT adjuvant as a component of oral live vaccines” as disclosed in the “BT lipopeptide oral adjuvant and vaccine composition” section of CN201810354453.2 was expressly precluded as a possible interpretation on two counts. First, live vaccines inherently contain various natural adjuvants, making exogenous adjuvants unnecessary. More critically, any added adjuvant must not adversely affect or modify the fundamental properties of the original vaccine. BT lipopeptides possess strong plasma membrane permeabilization effects that can inactivate enveloped viruses like Newcastle disease virus and their attenuated live vaccines. Therefore, the observation that oral administration of BT lipopeptides “mistakenly” mixed with attenuated live enveloped virus vaccines did not cause immunization failure was truly unexpected.

### Example 2

To validate and analyze the “BT Lipopeptide-Inactivated Oral Vaccine of Newcastle Disease” shown in Example 1, a multi-comparison trial was conducted (Table 2). One hundred chicks were divided into five different immunization treatment groups with 20 chicks each. Treatment group 1 served as the negative control group, receiving no vaccination but being orally administered 1 ml/animal of physiological saline at 2 days of age. Treatment group 2 served as the positive control group, receiving oral administration of 1.5 doses/animal of live attenuated Newcastle disease vaccine (La Sota strain, Xinpike, Harbin Pharmaceutical Group Biological Vaccine Co., Ltd.) at 2 days of age. Treatment groups 3-5 received orally administered inactivated Newcastle disease vaccine preparations: treatment group 3 received lipopeptide-singly-inactivated vaccine (lipopeptide+La Sota strain), while treatment groups 4 and 5 received detergent-plus-lipopeptide-dually-inactivated vaccine (detergent+lipopeptide+La Sota strain). The inoculum for treatment group 3 was prepared by mixing BT lipopeptides with the aforementioned attenuated live vaccine to form an aqueous solution containing 1000 ppm BT lipopeptides and 1.5 doses of inactivated vaccine per ml, administered orally at 1 ml/animal at 2 days of age. The inoculum for treatment group 4 consisted of Triton X-100 detergent, BT lipopeptides and the attenuated live vaccine mixed to form an aqueous solution containing 1000 ppm BT lipopeptides, 2% Triton X-100 and 1.5 doses of inactivated vaccine per ml, administered orally at 1 ml/animal at 2 days of age. The inoculum for treatment group 5 contained Triton X-100 detergent, BT lipopeptides and the attenuated live vaccine mixed to form an aqueous solution with 1000 ppm BT lipopeptides, 2% Triton X-100 and 4.5 doses of inactivated vaccine per ml, administered orally at 1 ml/animal at 2 days of age. It should be noted that the BT lipopeptides in the orally administered inocula of treatment groups 3-5 served not only as inactivating agents for vaccine preparation but also as vaccine adjuvants. Newcastle disease antibody titers were determined according to the Chinese National Standard GB/T16550-2020, while Newcastle disease vaccine viral content was measured following the “Determination of Median Lethal Dose and Infectious Dose (LD₅₀, ELD₅₀, ID₅₀, EID₅₀, TCID₅₀)” described in the Chinese Veterinary Pharmacopoeia (2010 Edition, Part III).

TABLE 2
Comparative test data on challenge with the Newcastle disease oral vaccine

| | Treatment Group Number | | | | |
|---|---|---|---|---|---|
| | 1 | 2 | 3 | 4 | 5 |
| Treatment | Without | Live | BT | BT + Triton | BT + Triton |
| | immunization | attenuated | inactivated | Inactivated | Inactivated |
| | | vaccine, | vaccine, oral | vaccine, oral | vaccine, oral |
| | | oral | | | |
| Method for Vaccine | — | — | 1000 ppm of | 1000 ppm of | 1000 ppm of |
| Inactivation | | | BT | BT | BT |
| | | | Lipopeptides | Lipopeptides + | Lipopeptides + |
| | | | | 2% Triton- | 2% Triton- |
| | | | | X100 | X100 |
| Content of Newcastle | — | >10⁸ | <1 | <1 | <1 |
| disease vaccine virus, | | | | | |
| EID₅₀/0.1 ml | | | | | |
| Dose of live Newcastle | 0 dose/ | 1.5 dose/ | 1.5 doses | 1.5 doses | 4.5 doses |
| disease vaccine | animal | animal | (inactivated)/ | (inactivated)/ | (inactivated)/ |
| gavaged at 2 days of | | | animal | animal | animal |
| age | | | | | |
| Mean antibody titer at | 0 | 6.81 | 6.38 | 0 | 0 |
| 25 days of age | | | | | |
| Survival rate, one-week | 0% | 95% | 90% | 0% | 0% |
| after the challenge with | | | | | |
| Texas GB strain at 28 | | | | | |
| days of age | | | | | |

The Newcastle disease antibody titer data at 25 days of age showed that the antibody levels of oral vaccination with BT lipopeptide-singly-inactivated vaccine (Treatment Group 3) were comparable to those of oral vaccination with attenuated live vaccine (Treatment Group 2). In contrast, the antibody levels of oral vaccination with dually-inactivated vaccine (Treatment Groups 4 and 5) were identical to those in the non-vaccinated group (Treatment Group 1), both of which were zero.

The classical highly pathogenic Texas GB strain is a standard challenge strain designated and provided by the USDA (U.S. Department of Agriculture) for testing the protection of Newcastle disease vaccines, and it was maintained in-house by the present inventor. All treatment groups were challenged with Texas GB strain at 28 days of age, and each chicken was injected with 10⁵ EID₅₀ of Texas GB virus at the chest muscle, and then observed for one week. The survival number of each treatment group was counted, and the survival rate of every group was calculated. The results showed that the protection of oral vaccination with BT lipopeptide-singly-inactivated vaccine (treatment group 3) was similar to that of oral vaccination with attenuated live vaccine (treatment group 2). However, the protection of oral vaccination with dually-inactivated vaccine (treatment groups 4 and 5) was the same as that without any vaccination (treatment group 1), both of which were zero.

The results demonstrated that the oral administration of the BT-lipopeptide-inactivated vaccine achieved comparable efficacy to the attenuated live vaccine, confirming the unexpected finding disclosed in Example 1. More significantly, incorporating detergent Triton X-100 as the second surfactant inactivator during the inactivation process completely abrogated the efficacy of BT-lipopeptide-inactivated oral vaccine, despite containing equivalent amounts of BT lipopeptide as vaccine adjuvant.

These findings indicated that the types of inactivator (inactivation method) and/or antigen morphology were critical for inactivated oral vaccines. Specifically, although both detergents and lipopeptides were surfactants, they disrupted phospholipid bilayers through fundamentally distinct mechanisms and with different results. Detergent surfactants such as Triton-X100 solubilize the phospholipid bilayer membranes into phospholipid micelles, causing the detachment and denaturation of integral membrane proteins from the phospholipids. In contrast, lipopeptides only permeabilize the phospholipid bilayers without significantly altering the structure of the phospholipid bilayer membranes and detaching the integral membrane proteins from the phospholipids, thereby maintaining the native conformations of the integral membrane proteins.

Like natural lipopeptide surfactants produced by other microorganisms, BT lipopeptides produced by B. texasporus are also non-ribosomal peptides. When orally administered, BT lipopeptides are not digested at all. Without wishing to be bound by theory, this may mean that BT lipopeptides are not only degraded by proteases, peptidases, or lipases in the gastrointestinal tract, but also inhibit the degradation of the perforated plasma membrane into which they are embedded and integral membrane proteins by proteases, peptidases, and lipases. That is to say, in the gastrointestinal tract, natural lipopeptide surfactants such as BT lipopeptides may have anti-digestive effects on the phospholipid bilayer membrane structure and the integral membrane proteins to which they bind. This is beneficial for the permeabilization of plasma membranes and the absorption and delivery of their integral membrane proteins by M cells. In contrast, the protein molecules that leak out due to plasma membrane permeabilization are completely “naked” and are naturally degraded by proteases in the gastrointestinal tract. Such differentiated digestion and degradation may actually play a role in the “post-inoculational purification” of the surface antigens of the pathogen's plasma membrane, which is conducive to the establishment of effective mucosal and systemic immunity with plasma-membrane-permeabilized and inactivated oral vaccines.

### Example 3

A conclusion or speculation from the analysis of the results of Example 2 was that an inactivated oral vaccine could be prepared by permeabilizing any pathogen having plasma membrane with a lipopeptide surfactant. In order to verify this speculation, the following trial by orally administering the inactivated Vibrio vaccine with BT lipopeptides was conducted.

Vibriosis is the number one disease in shrimp farming. Although the research on the inactivated oral vaccine of Vibrio parahaemolyticus has been going on for more than 30 years, there is currently still no practical vaccine available for shrimp farming. The currently existing oral “vaccines” of Vibrio, such as the high-temperature inactivated Vibrio vaccines, usually need to be added to the feed for long-term continuous feeding. No oral vaccine that meets the conventional definition of vaccines and stimulates the generation of protective immunity with a single dose has been disclosed or reported. Unlike vertebrates, shrimps do not have a classical specific immune system that produces stable immunoglobulin specificity through DNA-level gene recombination. Currently, only a “quasi-specific immune system” that produces temporary immunoglobulin specificity through mRNA alternative splicing has been found. Therefore, no matter what dosage form or delivery method is used, it may be more difficult to establish specific immunity with shrimp vaccines, and the duration of the protection “memory” may be shorter. In view of this, shrimp vibriosis has become a touchstone for further verifying inactivated oral vaccine technology with the plasma membrane permeabilized with lipopeptide.

Firstly, the plasma membrane-permeabilized inactivated vaccine of pathogenic Vibrio parahaemolyticus was prepared with BT lipopeptides. The traditional high-temperature inactivated vaccine of Vibrio parahaemolyticus was used as a control to compare the protection against challenge in Penaeus vannamei 10 days after single-dose inoculation. The pathogenic Vibrio parahaemolyticus used for vaccine preparation and challenge was donated by Demin Zhang and Haipeng Guo of Ningbo University. It was isolated from dead shrimps with Vibriosis in Ningbo, Zhejiang Province. This strain carries the pirAB toxin gene, and its pathogenicity has been verified by laboratory challenge test (any pathogenic Vibrio parahaemolyticus meeting the pathogenic requirements may be used).

Vaccine preparation: An aqueous suspension of Vibrio parahaemolyticus with a concentration of 1×10⁹ cfu/ml was treated at 100 °C for 15 minutes, and the number of live bacteria after the treatment was verified to be <10¹ cfu/ml. The aqueous suspension was made into a high-temperature inactivated vaccine containing 10⁹ dead bacteria/ml, which was packaged into vials of 50 ml each and then frozen and stored at −20 °C. An aqueous suspension of Vibrio parahaemolyticus bacterial with a concentration of 3×10⁹ cfu/ml was mixed with an aqueous solution of BT lipopeptides with a concentration of 1,500 ppm at a volume ratio of 1:2. The number of live bacteria after mixing was verified to be <10¹ cfu/ml. The solution was made into a BT-lipopeptide-inactivated vaccine containing 10⁹ dead bacteria/ml and 1,000 ppm BT lipopeptides, which was packaged into vials of 50 ml each and then frozen and stored at −20 °C.

Feed loading: the above two frozen vaccines were taken separately, and thawed at room temperature in a water bath. Then they were sprayed on the surface of No. 05 Prawn Compound Feed (Wuxi Tongwei Biotechnology Co., Ltd.) at a weight ratio of 1:10, stirred for 5 minutes, and air-dried to make high-temperature-inactivated vaccine loaded feed containing 108 dead bacteria/ml and BT-lipopeptide-inactivated vaccine loaded feed containing 108 dead bacteria/ml and 100 ppm BT lipopeptides. The aqueous solution of 1500 ppm BT lipopeptides was taken and sprayed on the surface of the above dried high-temperature-inactivated vaccine loaded feed at a weight ratio of 1:15. The feed were stirred for 5 minutes, and then air-dried to make the feed loaded with the BT-adjuvant-containing high-temperature-inactivated vaccine, which contains 100 ppm BT lipopeptides and 108 dead bacteria/ml.

Oral immunization: On the first day of the test, Penaeus vannamei with an average body length of approximately 50 mm in each treatment group were fed the unloaded and vaccine-loaded feeds listed in Table 3, and starting from the second day, the feeds provided to each group were unloaded feeds. The specific feeding method of Prawn Compound Feed followed the product instructions of the supplier (No. 05 Prawn Compound Feed; Wuxi Tongwei Biotechnology Co., Ltd.).

Challenge by Injection: On Day 11, the bacterial solution of the pathogenic Vibrio parahaemolyticus strain used to prepare the above inactivated vaccine was used for the challenge experiment by injection, with the target challenge intensity corresponding to 50% mortality within 48 hours. Feeding was stopped for two days, and normal feeding was resumed on Day 13. The number of survivors was determined on Day 21 to calculate survival rate and relative survival rate (RSP=(the survival rate of the treatment group−the survival rate of the control group without immunization but challenged)/(the survival rate of the control group without immunization or challenge−the survival rate of the control group without immunization but challenged)).

TABLE 3
Comparative test data of protection of single-dose oral inoculation of BT-
lipopeptide-inactivated vaccine and high-temperature-inactivated vaccine

| | Treatment Group Number | | | | |
|---|---|---|---|---|---|
| | 1 | 2 | 3 | 4 | 5 |
| treatment | Without | Without | Immunized | Immunized | BT- |
| | immunization | immunization, | with high- | with BT | inactivated |
| | or challenge | but challenged | temperature- | adjuvant + | vaccine, |
| | | | inactivated | high | and |
| | | | vaccine, and | temperature | challenged |
| | | | challenged | inactivated | |
| | | | | vaccine and | |
| | | | | challenged | |
| Day 1, oral | — | — | High- | 100 ppm BT | BT- |
| immunization: | | | temperature- | adjuvant | lipopeptide- |
| feed-loaded | | | inactivated | High- | inactivated |
| inactivated | | | vaccine | temperature- | vaccine |
| Vibrio vaccine | | | | inactivated | |
| | | | | vaccine | |
| Day 11, Vibrio | − | + | + | + | + |
| challenge by | | | | | |
| injection | | | | | |
| Relative | 100% | 0% | 0% | 0% | 43% |
| survival rate on | | | | | |
| Day 21 | | | | | |

On Day 21, i.e., Day 10 after challenge, the mortality rate of each treatment group was calculated and the relative survival rate of each group was calculated. The relative survival rate of treatment group 5 with oral vaccination with a single dose of BT-lipopeptide-inactivated vaccine exceeded 40%. In contrast, the relative survival rate of treatment group 3 with oral vaccination with a single dose of high-temperature-inactivated vaccine was zero. The relative survival rate of treatment group 4, orally vaccinated with a single dose of high-temperature-inactivated vaccine plus 100 ppm BT lipopeptides as an adjuvant, was also zero. These results showed that the single-dose vaccination of BT-lipopeptide-inactivated vaccine had much higher protection than the single-dose vaccination of high-temperature-inactivated vaccine, and addition of BT lipopeptides as an adjuvant failed to increase protection of the high-temperature-inactivated vaccine. This once again showed that the means of inactivation and antigen morphology are critical for inactivated oral vaccines.

### Example 4

Although the Vibrio BT-lipopeptide-inactivated oral vaccine disclosed in Example 3 represented a great step forward in the practicality of prawn Vibrio vaccines, its protection was still insufficient. In view of the effects of BT lipopeptides themselves on modulating non-specific immunity and promoting animal health and growth, the following combination comparison test (Table 4) was conducted to examine the protection against challenge in the combination regimen of “single dose vaccination of BT-lipopeptide-inactivated Vibrio vaccine+continuous feeding of BT peptides”.

The methods for vaccine preparation, feed loading, oral inoculation, and challenge by injection in this Example were the same as those in Example 3. The aqueous solution of 1,500 ppm BT lipopeptides was sprayed on the feed at a ratio of 1:7.5, stirred evenly, and then dried, so as to prepare the feed containing 200 ppm BT lipopeptides. The body length gain and body weight gain of survivors were measured for each treatment on Days 1 and 21, and the number of survivors was determined on Day 21 to calculate the relative survival rate.

On Day 21 (day 10 post-challenge), the relative survival rate of treatment group 4, which was immunized only with BT-lipopeptide-inactivated vaccine but for which BT lipopeptides were not added to the feed, was 38%. This result was similar to that in Example 3, i.e., showing only partial protection. The relative survival rate of treatment group 3, for which only BT lipopeptides were added to the feed without immunization, was 40%, also showing only partial protection. However, treatment group 5, which was both immunized and had BT lipopeptides added to the feed, showed a relative survival rate of 99%, showing nearly complete protection against challenge.

TABLE 4
Combined comparison test data of BT-inactivated vaccine and BT feed additive

| | Treatment Group Number | | | | |
|---|---|---|---|---|---|
| | 1 | 2 | 3 | 4 | 5 |
| treatment | Without | Without | Without | With | With |
| | immunization, | immunization, | immunization, | immunization, | immunization, |
| | without | without | with additive, | without | with additive, |
| | additive, | additive, | and | additive, | and |
| | without | but challenged | challenged | but challenged | challenged |
| | challenge | | | | |
| On Day 1, oral | − | − | − | + | + |
| immunization: | | | | | |
| the feed | | | | | |
| containing | | | | | |
| inactivated | | | | | |
| Vibrio BT | | | | | |
| vaccine was fed | | | | | |
| From Days 2 | − | − | + | − | + |
| to 10 and 13 | | | | | |
| to 21, the feed | | | | | |
| containing | | | | | |
| 200 ppm BT | | | | | |
| lipopeptides | | | | | |
| was continuously | | | | | |
| fed. | | | | | |
| On Day 11, | − | + | + | + | + |
| Vibrio | | | | | |
| challenge by | | | | | |
| injection | | | | | |
| Relative | 100% | 0% | 40% | 38% | 99% |
| survival rate | | | | | |
| on Day 21 | | | | | |
| Average body | 10.6 | 7.3 | 7.0 | 9.5 | 10.5 |
| length gain on | | | | | |
| Days 1-21 | | | | | |
| (mm per | | | | | |
| shrimp) | | | | | |
| Average body | 0.8 | 0.3 | 0.4 | 0.6 | 0.8 |
| weight gain | | | | | |
| on Days 1-21 | | | | | |
| (g per shrimp) | | | | | |

Under equivalent challenge conditions, oral immunization of the BT-lipopeptide-inactivated vaccine showed a growth-promoting function, and the body length gain and body weight gain of survivors in immunized treatment groups 4 and 5 were substantially better than those in unimmunized treatment groups 2 and 3.

Under equivalent immunization and challenge conditions, continuous feeding with BT lipopeptides showed a growth-promoting effect, and the survivors of treatment group 5 fed with the feed containing 200 ppm BT lipopeptides showed better body length gain and body weight gain than treatment group 4, which was not fed with feed containing 200 ppm BT lipopeptides.

### Example 5

The combination regimen of “single dose vaccination of BT-lipopeptide-inactivated Vibrio vaccine+continuous feeding of BT peptides” in Example 4 showed the effect of significantly promoting shrimp growth in the laboratory Vibrio challenge test. However, the practicality of the combination regimen deserves further discussion, especially regarding the need for improvement in the following two aspects. Firstly, continuous feeding with BT peptides involves cost issues of use. Secondly, clinical vibriosis does not occur every time under field production conditions. Therefore, it is necessary to test whether the combination regimen of “single-dose vaccination of BT-lipopeptide-inactivated Vibrio vaccine+pulse feeding of BT peptides” also has a significant growth-promoting effect in the absence of clinical vibriosis.

The field experiment was carried out in a shrimp farm in Ningbo, which had small shed earth ponds. A total of 37 sheds within the same batch and under the same conditions were divided into two treatment groups. The control group (20 sheds) was neither immunized with BT-lipopeptide-inactivated vaccine nor treated with BT peptides, and only No. 05 Prawn Compound Feed was given. On Day 1 of the experiment, the experimental group (17 sheds) was fed with the feed loaded with BT-lipopeptide-inactivated vaccine (with a number of 108 dead Vibrio per gram)+200 ppm BT peptides. To reduce the amount of BT peptides used, the feed loaded with 200 ppm of BT peptides was fed only on Day 8, i.e., BT peptides were no longer used continuously. Shrimps (10 shrimps per shed) were collected on Days 1, 8, and 15, then the body length of the collected shrimps was measured, and the average value of the treatment groups was calculated. No clinical vibriosis occurred during the experiment.

TABLE 5
growth experiment data of prawns in field experiment
with simplified “BT-lipopeptide-inactivated
vaccine + BT peptides combination regimen”

| | Treatment Group | | | |
|---|---|---|---|---|
| | Control group | | Experimental group | |
| | Feed | Average | | Average |
| | supplemen- | body | | body |
| Day | tation | length | Feed supplementation | length |
| 1(P31) | — | 5.2 cm | BT-lipopeptide-inactivated | 5.2 cm |
| | | | vaccine (with a number of | |
| | | | 108 dead Vibrio per gram)) + | |
| | | | 200 ppm BT peptide | |
| 8(P38) | — | 6.1 cm | 200 Ppm BT peptide | 6.4 cm |
| 15(P45) | | 7.0 cm | | 7.5 cm |

The data in Table 5 showed that the body length of prawns in the experimental group significantly surpassed that of the control group on Day 8, and the superiority of body length in the experimental group, which was already evident on Day 8, further expanded and became more pronounced during the subsequent week. These results from the field experiment showed that the combination regimen of “single-dose vaccination of BT-lipopeptide-inactivated Vibrio vaccine+pulse feeding of BT peptides” significantly promoted the growth of shrimps, regardless of whether clinical vibriosis occurred.

Another pilot experiment was also carried out in this field: 31 sheds within the same batch of the field and under the same conditions were divided into two treatment groups. The control group (20 sheds) was neither immunized nor received BT peptides, and only No. 05 Prawn Compound Feed was given. On P31 (post-larvae Day 31), P45, P59, P73, P87, and P101, the Penaeus vannamei in the experimental group (11 sheds) were fed with feed loaded with BT-lipopeptide-inactivated vaccine (with a number of 108 dead Vibrio per gram)+200 ppm BT peptides. A feed loaded with 200 ppm of BT peptides was fed on P38, P52, P66, P80, P94, and P108, i.e., BT peptides were no longer used continuously. If there is vibriosis, a large number of shrimps will die; thus, this shed will be counted as a diseased shed. The number of shrimps in each shed was counted at the harvest time (approximately at post-larvae Day 120). The survival rates and yields were compared between the two groups. Success rate=the number of disease-free sheds in the treatment group throughout the course/the total number of sheds in the treatment group, survival rate=the number of shrimps harvested in each shed/the number of starting young shrimps.

TABLE 6
Experiment data of prawns in a field experiment
with “BT-lipopeptide-inactivated vaccine +
alternating pulse BT peptides” combination regimen

Treatment Group

| | Control group | Experimental group |
|---|---|---|
| | (20 sheds) | (11 sheds) |
| Success Rate (%) | 65 | 100 |
| Survival rate (%) | 42.7 +/− 29.7 | 76.3 +/− 16.5 |
| Yield (kg) | 328.3 +/− 227.3 | 574.0 +/− 160.4 |

The data in Table 6 showed that all indicators in the experimental group were higher than those in the control group. Among these indicators, the difference in yield was extremely significant (P<0.01) and the difference in survival rate was significant (P<0.05). The results of the pilot field experiment showed that the combination regimen of “single-dose vaccination of BT-lipopeptide-inactivated Vibrio vaccine+alternating pulse feeding of BT peptides” significantly improved the survival rate and yield of shrimps.

### Example 6

Although the method of Example 4 was simplified to some extent, Example 5 still involved two preparations, which may lead to operational errors due to confusion during the manufacturing or application process. Thus, it was necessary to further simplify the process and try to combine the two preparations into a single preparation. This Example was conducted according to the settings of the experimental sites and shrimp farms shown in Table 6, wherein “relative unit yield”=yield per unit area of the experimental group/yield per unit area of the control group.

The tested single preparation comprised BT-lipopeptide-inactivated vaccine (with a number of 109 dead Vibrio per gram)+BT peptides (2,000 ppm). For the experimental group, feed loaded with BT-lipopeptide-inactivated vaccine (with a number of 108 dead Vibrio per gram)+200 ppm BT peptides was fed at P35, P45, P55, and P65, with each feeding amount being ¼ of the daily amount.

The control group was fed with feed, and also with freeze-dried powder of Bdellovibrio (“Vibrio-Hunting Hero”, Guangzhou Shunzhou Biotechnology Co., Ltd.) or functional feed of Chinese herbal medicine formula (“Xiayoukang”, Ningbo Sansheng Biotechnology Co., Ltd.). The feeding methods of freeze-dried powder of Bdellovibrio and functional feed of Chinese herbal medicine formula followed the product instructions.

At the time of harvest (about P120), the yield per unit area of the experimental group, the yield per unit area of the control group, and the number of sheds without disease within the whole course were calculated.

Table 7 presents a summary of the field comparative experiments of different modes of farming and production, and the results showed that the use of the aforementioned single preparation can also effectively improve the production indicators. In these field experiments, it is worth noting that the scale of the control group was at least twice that of the experimental group, which further validates the significance of the results.

TABLE 7
summary of results in a prawn field experiment with the single preparation regimen
of “BT-lipopeptide-inactivated vaccine + BT peptides”

| | | Shrimp | Experimental | | |
|---|---|---|---|---|---|
| | | farm | group | Control group | |
| Month | Location | Mode | Scale | Anti-Vibrio products | Result |
| March to | Cixi, | Small- | 8 million | Freeze-dried powder of | Success rate: |
| June | Zhejiang | Shed, | shrimps | Bdellovibrio (“Vibrio- | Experimental group 100% |
| | | Earth | | Hunting Hero”, | VS. control group 44% |
| | | pond | | Guangzhou Shunzhou | |
| June to | Rudong, | Factory | 1.5 million | Biotechnology Co., | Success rate: |
| September | Jiangsu | | shrimps | Ltd.) was splashed on | Experimental group 100% |
| | | | | water | VS. control group 25% |
| February | Xiaoshan, | Large- | 2 million | Functional feed of | Relative unit yield: |
| to May | Zhejiang | shed, | shrimps | Chinese herbal medicine | Experimental group 112% |
| | | Earth | | formula (“Xiayoukang”, | VS. control group 100% |
| | | pond | | Ningbo Sansheng | |
| June to | Xiaoshan, | Large- | 8 million | Biotechnology Co., | Relative unit yield: |
| September | Zhejiang | shed, | shrimps | Ltd.) was fed once every | Experimental group 133% |
| | | Earth | | four meals | VS. control group 100% |
| | | pond | | | |

Although the present disclosure has been described with reference to illustrative embodiments, those skilled in the art will understand that various other changes, omissions and/or additions may be made and that elements of the described embodiments may be replaced with substantial equivalents without departing from the spirit and scope of the present disclosure. In addition, many modifications can be made to adapt particular situations or materials to the teachings of the present disclosure without departing from the scope of the present disclosure. Accordingly, it is not intended herein to limit the invention to the specific embodiments disclosed for carrying out the invention, but rather it is intended that the invention will include all embodiments falling within the scope of the appended claims.

1. An inactivated vaccine comprising a pathogen or a live vaccine thereof inactivated by permeabilization of the plasma membrane with a permeabilizer, wherein the inactivated vaccine comprises a permeabilized plasma membrane and its integral membrane protein, wherein the pathogen is a pathogen comprising a plasma membrane, and the permeabilizer comprises a lipopeptide as a surfactant.

2. The inactivated vaccine according to claim 1, wherein the lipopeptide comprises a natural lipopeptide and/or an artificially synthesized lipopeptide.

3. The inactivated vaccine according to claim 1, comprising a pharmaceutically acceptable carrier, or an adjuvant.

4. A method for preparing the inactivated vaccine of claim 1, comprising:
inactivating the pathogen or the live vaccine thereof by permeabilizing its plasma membrane with a permeabilizer, wherein the permeabilizer comprises a lipopeptide as a surfactant.

5. An oral composition comprising an inactivated vaccine of claim 1.

6. Use of a permeabilizer in the manufacture of the inactivated oral vaccine of claim 1, which is accomplished by perforating the plasma membrane of the pathogen or the live vaccine thereof to cause exudation of the contents inside the plasma membrane, thus inactivating the pathogen or live vaccine thereof; wherein the permeabilizer comprises a lipopeptide as a surfactant.

7.-10. (canceled)

11. The inactivated vaccine of claim 1, wherein the pathogen is selected from the group consisting of an enveloped virus, a bacterium, a fungus, an endoparasite and a cancer cell.

12. The inactivated vaccine of claim 1, wherein the vaccine further comprises exuded contents from the pathogen or the live vaccine thereof after the permeabilization.

13. The inactivated vaccine of claim 1, wherein the vaccine is inactivated by a step comprising plasma membrane-permeabilization of the pathogen or the live vaccine thereof such that the number of live pathogens is less than 10¹/ml and/or the number of dead pathogens is greater than 10⁹/ml; and/or
the inactivated oral vaccine is an oral solution, a powder, a tablet, a capsule, a drench solution or an oral bolus solution; and/or
the inactivated oral vaccine is a vaccine administered by direct oral administration, water-based delivery, feed-based delivery, gavage, or oral bolus injection; and/or
the permeabilizer contains no surfactants except the lipopeptide.

14. The inactivated vaccine of claim 2, wherein the natural lipopeptide is selected from the group consisting of Surfactins, Fengycins, Iturins, Daptomycin, Polymyxins, Viscosin, and BT lipopeptide produced from Brevibacillus texasporus.

15. The inactivated vaccine of claim 2, wherein the BT lipopeptide produced from B. texasporus comprises a lipopeptide having the sequence set forth in any one of SEQ ID NOs: 1-9 or any combination thereof; and/or
the pathogen is selected from the group consisting of: Coronaviridae, Influenza viruses, Paramyxoviridae, Herpesviridae, Poxviridae, Hepadnaviridae, Baculoviruidae, Retroviridae, African swine fever virus, Acinetobacter, Bacilli, Borrelia, Campylobacter, Clostridia, Vibrio, E. coli, Enterococcus, Neisseria, Haemophilus, Mannheimia, Mycobacteria, Mycoplasma, Pasteurellaceae, Pseudomonas, Salmonella, Staphylococcus, Streptococcus, Coccidia, Nematoda, Cestoda, and cancer cells; and/or
the pathogen is selected from the group consisting of Newcastle disease virus and Vibrio parahaemolyticus.

16. The method of claim 4, wherein the method does not comprise a step of isolating an antigen and/or a step of artificially encapsulating an antigen; and/or
when the pathogen is inactivated, the method does not comprise inactivation means other than permeabilization; and/or
the permeabilizer contains no surfactants except the lipopeptide; and/or
plasma membrane of the pathogen or the live vaccine thereof is permeabilized, such that the number of live pathogens is less than 10¹/ml and/or the number of dead pathogens is greater than 10⁹/ml; and/or
the lipopeptide comprises a natural lipopeptide and/or an artificially synthesized lipopeptide; and/or
the natural lipopeptide is selected from the group consisting of Surfactins, Fengycins, Iturins, Daptomycin, Polymyxins, Viscosin, and BT lipopeptide produced from B. texasporus; and/or
the BT lipopeptide produced from B. texasporus comprises a lipopeptide(s) having the sequence set forth in any one of SEQ ID NOs: 1-9 or any combination thereof.

17. The oral composition of claim 5, wherein the oral composition comprises food, drinking water, or beverage; and/or
the food is selected from the group consisting of a milk substitute, growth feed, finisher feed, pre-starter feed, and starter feed; and/or
the composition comprises one or more of cereal, soybean flour, isolated soybean protein, soybean oil, soybean fat, skim milk, fish meal, meat meal, bone meal, blood meal, plasma protein, whey, rice bran, wheat bran, sweeteners, minerals, vitamins, and salts; and/or
the cereal is selected from the group consisting of barley, soybean, wheat, triticale, rye, corn, and rice; and/or
the oral composition comprises an ingredient that modulates immunity or promotes health or growth.

18. The oral composition of claim 17, wherein the ingredient is BT lipopeptide which is either mixed with or separated from the inactivated vaccine.

19. The oral composition of claim 18, wherein the BT lipopeptide is BT lipopeptide produced from B. texasporus or BT lipopeptide having the sequence set forth in any one of SEQ ID NOs: 1-9 or any combination thereof.

20. A method for treating and/or preventing an infection by a pathogen or a disease caused by a pathogen in a subject, comprising administrating to the subject the inactivated vaccine of claim 1.

21. The method of claim 20, wherein the infection or disease is selected from the group consisting of influenza, viral hepatitis, African swine fever, porcine reproductive and respiratory syndrome, colibacillosis, salmonellosis, necrotic enteritis, vibrosis, bovine shipping fever pneumonia, Gonorrhea, urinary tract infection, coccidiosis, nematodiasis, cestodiasis, and cancer; and/or
the pathogen is selected from the group consisting of Newcastle disease virus and Vibrio parahaemolyticus; and/or
the subject is a bird, a livestock, an aquatic animal, or a companion animal.

22. A method for improving bodyweight gain and/or food conversion in a subject, comprising administrating to the subject of the inactivated vaccine of claim 1.

23. The method of claim 22, wherein the subject is a bird, a livestock, an aquatic animal, or a companion animal and/or the inactivated vaccine is administrated by in ovo feeding, spraying in the form of droplets onto a bird or immersion of an aquatic animal.

24. The use of claim 6, wherein the lipopeptide comprises a natural lipopeptide and/or an artificially synthesized lipopeptide; and/or
the natural lipopeptide is selected from the group consisting of Surfactins, Fengycins, Iturins, Daptomycin, Polymyxins, Viscosin, and lipopeptides produced from B. texasporus; and/or
the lipopeptides produced from B. texasporus comprise a lipopeptide having the sequence set forth in any one of SEQ ID NOs: 1-9 or any combination thereof.


SEQUENCE LISTING (9 sequences)

SEQ ID NO: 1 - protein, 14 aa, synthetic construct
  REGION 1..14: note = Peptide
  SITE 1: note = MISC_FEATURE - 2-hydroxy-3-methylpentanoic acid
  SITE 2: note = MISC_FEATURE - 2,3-dehydro-2-aminobutyric acid
  SITE 4: note = MISC_FEATURE - ornithine
  SITE 14: note = MISC_FEATURE - valinol
XXLXIVVKVLKYLX

SEQ ID NO: 2 - protein, 14 aa, synthetic construct
  REGION 1..14: note = Peptide
  SITE 1: note = MISC_FEATURE - 2-hydroxy-3-methylpentanoic acid
  SITE 2: note = MISC_FEATURE - 2,3-dehydro-2-aminobutyric acid
  SITE 4: note = MISC_FEATURE - ornithine
  SITE 14: note = MISC_FEATURE - valinol
XXMXIVVKVLKYLX

SEQ ID NO: 3 - protein, 14 aa, synthetic construct
  REGION 1..14: note = Peptide
  SITE 1: note = MISC_FEATURE - 2-hydroxy-3-methylpentanoic acid
  SITE 2: note = MISC_FEATURE - 2,3-dehydro-2-aminobutyric acid
  SITE 4: note = MISC_FEATURE - ornithine
  SITE 14: note = MISC_FEATURE - valinol
XXVXIVVKVLKYLX

SEQ ID NO: 4 - protein, 14 aa, synthetic construct
  REGION 1..14: note = Peptide
  SITE 1: note = MISC_FEATURE - 2-hydroxy-3-methylpentanoic acid
  SITE 2: note = MISC_FEATURE - 2,3-dehydro-2-aminobutyric acid
  SITE 4: note = MISC_FEATURE - ornithine
  SITE 14: note = MISC_FEATURE - valinol
XXLXIIVKVLKYLX

SEQ ID NO: 5 - protein, 14 aa, synthetic construct
  REGION 1..14: note = Peptide
  SITE 1: note = MISC_FEATURE - 2-hydroxy-3-methylpentanoic acid
  SITE 2: note = MISC_FEATURE - 2,3-dehydro-2-aminobutyric acid
  SITE 14: note = MISC_FEATURE - valinol
XXLKIIVKVLKYLX

SEQ ID NO: 6 - protein, 14 aa, synthetic construct
  REGION 1..14: note = Peptide
  SITE 1: note = MISC_FEATURE - 2-hydroxy-3-methylpentanoic acid
  SITE 2: note = MISC_FEATURE - 2,3-dehydro-2-aminobutyric acid
  SITE 4: note = MISC_FEATURE - ornithine
  SITE 14: note = MISC_FEATURE - valinol
XXVXVVVKVLKYLX

SEQ ID NO: 7 - protein, 14 aa, synthetic construct
  REGION 1..14: note = Peptide
  SITE 1: note = MISC_FEATURE - 2-hydroxy-3-methylpentanoic acid
  SITE 2: note = MISC_FEATURE - 2,3-dehydro-2-aminobutyric acid
  SITE 4: note = MISC_FEATURE - ornithine
  SITE 14: note = MISC_FEATURE - valinol
XXMXIIVKVLKYLX

SEQ ID NO: 8 - protein, 14 aa, synthetic construct
  REGION 1..14: note = Peptide
  SITE 1: note = MISC_FEATURE - 2-hydroxy-3-methylpentanoic acid
  SITE 2: note = MISC_FEATURE - 2,3-dehydro-2-aminobutyric acid
  SITE 4: note = MISC_FEATURE - ornithine
  SITE 14: note = MISC_FEATURE - valinol
XXIXIVVKVLKYLX

SEQ ID NO: 9 - protein, 14 aa, synthetic construct
  REGION 1..14: note = Peptide
  SITE 1: note = MISC_FEATURE - 2-hydroxy-3-methylpentanoic acid
  SITE 2: note = MISC_FEATURE - 2,3-dehydro-2-aminobutyric acid
  SITE 4: note = MISC_FEATURE - ornithine
  SITE 14: note = MISC_FEATURE - valinol
XXFXIVVKVLKYLX